(19) United States
(12) Patent Application Publication
(10) Pub. No.: US 2006/0155235 A1
(43) Pub. Date: Jul. 13, 2006

(54) Hemostatic compression bandage

(76) Inventor: Evelyn S. Sawyer, Freeport, ME (US)

Correspondence Address: IP STRATEGIES, 12 1/2 WALL STREET, SUITE I, ASHEVILLE, NC, 28801, US

(21) Appl. No.: 11/305,894

(22) Filed: Dec. 16, 2005

Related U.S. Application Data
(60) Provisional application No. 60/637,222, filed on Dec. 17, 2004.

(51) Int. Cl. A61F 13/00

(52) U.S. Cl. 602/48

(57) ABSTRACT

A hemostatic compression bandage includes a flexible backing element, a powdered hemostatic substance, and a flexible film element. The backing element includes a first containment region. The film element includes a second containment region corresponding to the first containment region. The film element is removably attached to the backing element so as to contain the hemostatic substance between the backing element and the film element within a volume defined by the first containment region and the second containment region. The backing element includes a pressure region located outside of the first containment region. The film element includes a pull-tab region located outside of the second containment region and at a first side of the bandage. The pull-tab region has sufficient length to be folded back across the second containment region and extending beyond a second side of the bandage that is opposite the first side of the bandage.

### FIELD OF THE INVENTION

The invention relates generally to hemostatic bandages useful for treating traumatic bleeding, particularly in military or civilian field (pre-hospital) settings.

### BACKGROUND OF THE INVENTION

From the Civil War until the present time, the largest single cause of death on the battlefield is uncontrolled bleeding. A similar problem exists in the civilian sector, such as when emergency personnel apply tourniquets or other standard physical methods of pressure to a bleeding wound. Although compression to the wound alone is effective in some cases, for arterial bleeding there is a clear benefit to the addition of a hemostatic agent to a compression bandage. The hemostatic substance should be delivered to the wound site in a form that permits maximum contact with the patient's blood, and therefore rapid hydration and activation of the substance. For military and emergency workers, a hemostatic bandage must also be suitable for field conditions and simple enough for use by those who lack medical training.

Hemostatic agents are well known to those in the art and include minerals (Alam et al. 2004), carbohydrates (Pusateri et al 2003), and proteins (Holcomb et al. 1999). In field situations, refrigeration, pre-hydration, and liquid delivery systems usually are not available, and therefore a consensus has developed for the use of dry hemostatic agents that can be carried in the field, and applied by non-medical personnel (Sondeen et al. 2003).

Sondeen et al (2003) compared different bandages made from dry hemostatic agents including alginates, chitosan, zeolite, collagen, and human fibrinogen and thrombin (fibrin), and found that only the fibrin dressing stopped bleeding in an otherwise fatal arterial injury.

To avoid the risks of exposure to infectious agents from human or mammalian plasma proteins, Wang et al (2000), Michaud et al (2001), and Sawyer et al (2001) developed fibrin sealants from the plasma of farmed salmon. In a pig aortotomy model, dressings containing these lyophilized salmon proteins were effective in treating this high-pressure, actively bleeding arterial injury (Rothwell et al. 2005).

Although a dry fibrin dressing is currently the goal for field hemostasis, the ideal delivery system or bandage has not been developed. This ideal system would include:
1. A backing that permits continuous, firm, pressure on the wound.
2. Delivery of the dry proteins in the most available form, that is, a loose, fine powder that provides maximum contact with the patient's blood.

One delivery approach is illustrated by “Quick Clot” (Z-Medica, Newington, Conn.). Pressure is first applied to the wound with whatever material (such as gauze) is available. A zeolite powder is poured into the wound and pressure is reapplied. This system fails 1 above, because the pressure on the wound is interrupted.

In a later modification by Z-Medica (Hursey, 2005), the hemostatic powder is enclosed in a mesh bag. Also, Bell et al. placed a hemostatic powder in an expandable pouch, separated from the wound by a blood-permeable or net-like film. These systems recognize the importance of the large surface area provided by use of a powder, but fail 2 above because the film slows and limits contact between the blood and the hemostatic substance.

A third approach is shown by Sawyer (1983), who developed a band-aid-like device for treating minor bleeding by lyophilizing collagen on a gauze pad with an adhesive strip backing. Lyophilizing a hemostatic substance in or on a backing was developed further by MacPhee, (2004a) in the American Red Cross bandage. Here the proteins are provided as a sandwich of human fibrinogen and thrombin lyophilized on several layers of backing. This system fails 2 above as it results in a hard, cardboard-like bandage that slows contact between the proteins and the patient's blood. Also, the proteins tend to “flake off” as the bandage is applied (Sondeen et al. 2003).

Since a finely ground powder offers a large surface area of the proteins to contact blood, MacPhee (2000, 2001, and 2004b) anticipates two methods of fixing this powder to a backing. In the first example, the proteins are mixed with a soluble adhesive substance and applied to the backing; this of course, delays contact with the blood. In the second example, the backing can be coated with an adhesive substance, and then the powder applied. This system would be satisfactory for small amounts of powder, but MacPhee acknowledges that the quantity of lyophilized fibrinogen and thrombin needed for hemostasis in a fibrin bandage produced a powder layer 0.5 cm thick. He suggests an impermeable film to hold the powder in place. This film is removed prior to application in which case, much of the powder could be lost before it reaches the wound site.

Rothwell et al. (2005) applies the hemostatic powder, lyophilized fibrinogen and thrombin, pressed on a gauze backing, directly to the wound. But even in the surgical setting, an unknown amount of the powder is lost before direct contact with the wound.

### BRIEF SUMMARY OF THE INVENTION

The present invention offers a solution to the described problems by providing a system that delivers a fine, soft, loose powder directly to the wound site, with no barrier between the powder and the wound, and no interruption of pressure to the wound.

Thus, the present invention permits continuous pressure on the wound. There is no need to release the pressure while a hemostatic powder is released into the wound. The present invention also delivers the hemostatic substance in its optimal form, that is, a loose, fine, powder that provides maximum surface area in contact with the patient's blood, resulting in rapid hydration and activation. The present invention also delivers the hemostatic powder directly to the wound without loss during the application process.

The present invention is a hemostatic bandage useful for treatment of traumatic bleeding, particularly when continuous pressure or compression and localized delivery of a hemostatic substance are beneficial. In particular, the bandage allows a hemostatic dry powder, such as a powder including salmon fibrinogen and thrombin, to be delivered to the wound site while firm pressure is being applied to the site.

According to an aspect of the invention, a hemostatic compression bandage includes a flexible backing element, a powdered hemostatic substance, and a flexible film element. The backing element includes a first containment region. The film element includes a second containment region corresponding to the first containment region. For example, the peripheries of the first and second containment regions can match, or the containment regions can otherwise overlap in area. Preferably, the containment areas are defined on the backing element and the film element. The film element is removably attached to the backing element so as to contain the hemostatic substance between the backing element and the film element within a volume defined by the first containment region and the second containment region, or by an overlap of these regions. The film element includes a pull-tab region located outside of the second containment region and at a first side of the bandage. The pull-tab region has sufficient length to be folded back across the second containment region to extend beyond a second side of the bandage.

The backing element can include a pressure region located outside of the first containment region on at least a first side of the bandage. For example, the pressure region can be located on a side of the bandage that is opposite the first side of the bandage, although other sides of the bandage can include the pressure region instead or in addition.

A method of treating an open wound using this bandage can include placing the bandage over the wound, with the film element pressed against the wound. Pressure can be applied against the backing element over the wound site. The pull-tab region of the film element can be grasped. The film element can be pulled out from under the powdered hemostatic substance and away from the backing element, allowing the powdered hemostatic substance to come into contact with the wound. If the bandage includes a pressure region of the backing element, pressure can continue to be applied against the backing element over the wound site, with particular pressure applied to the pressure region in an amount sufficient to hold the bandage in place while the pull-tab region is pulled. Pressure can then continue to be applied against the backing element until the powdered hemostatic substance causes blood at the surface of the wound to clot. Pressure can be applied continuously to the wound site throughout this process.

The backing element can be fabricated from a non-bioabsorbable material. For example, the backing element can include at least one of an alginate such as calcium alginate, silicone, plastic, and cotton gauze. Alternatively, the backing element can be fabricated from a non-toxic bioabsorbable material. For example, the backing element can include at least one of a glycolic acid polymer and a glycolic acid/lactic acid copolymer. As another alternative, the backing element can include polyester felt.

The hemostatic substance can include a protein. For example, the hemostatic substance can include at least one of fibrinogen and thrombin, such as at least one of lyophilized salmon fibrinogen and thrombin. Alternatively, or in addition, the hemostatic substance can include a carbohydrate. For example, the hemostatic substance can include at least one of an algal polymer, such as an algae-derived polysaccharide, and chitosan. Alternatively, or in addition, the hemostatic substance can include a mineral. The hemostatic compression bandage can also include powdered calcium, added to the hemostatic substance.

The film element can include plastic. For example, the film element can include polyethylene. The film element can be removably attached to the backing element by a seal that defines a perimeter of the first containment region and the second containment region. For example, the seal can include an adhesive substance. Alternatively, the seal can be a bond formed by at least one of heat and pressure.

The backing element, the powdered hemostatic substance, and the film element preferably are fabricated from materials that are suitable for sterilization, for example, by gamma radiation.

The backing element can include an adhesive substance around a periphery, facing the film element and outside of an outer periphery of the film element. A method of treating an open wound with this bandage, for example, can include placing the bandage over the wound, with the film element pressed against the wound. Pressure can be applied against the backing element over the wound site. The pull-tab region of the film element can be grasped. The film element can be pulled out from under the powdered hemostatic substance and away from the backing element, allowing the powdered hemostatic substance to come into contact with the wound. If the bandage includes a pressure region of the backing element, pressure can continue to be applied against the backing element over the wound site, with particular pressure applied to the pressure region in an amount sufficient to hold the bandage in place while the pull-tab region is pulled. The adhesive substance can be pressed against skin around the wound, thereby adhering the backing element to the skin. Pressure can be applied continuously to the wound site throughout this process.

### BRIEF DESCRIPTION OF THE DRAWING

FIG. 1 shows an exploded view of an exemplary bandage according to the invention.

### DETAILED DESCRIPTION OF THE INVENTION

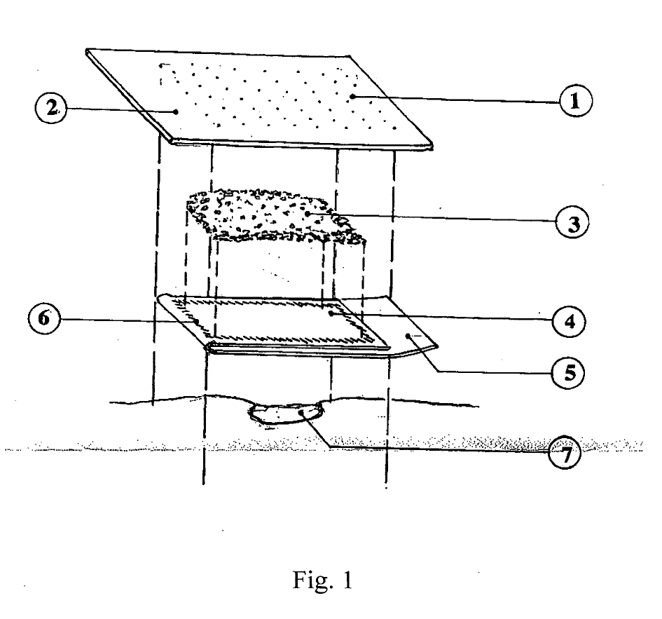

As shown in FIG. 1, an exemplary embodiment of the bandage of the present invention includes a backing element 1 having at least one end serving as a pressure area or region 2. The exemplary bandage also includes a powdered hemostatic substance 3 disposed between the backing element 1 and a removable, protective film element 4 that is folded back on itself. The film element 4 includes a pull-tab region 5 extending beyond an edge of the bandage. An adhesive, heat, or pressure seal 6 around a containment region for the powdered hemostatic substance 3 removably attaches the backing element 1 to the protective film element 4, holding the powdered hemostatic substance 3 in place between the two elements 1, 4. The pressure region 2 extends beyond the containment region on at least one side of the bandage.

The bandage is applied to a wound area by placing the protective film against the wound 7. Pressure, for example, hand pressure, is applied to the backing against the wound 7. Particular pressure is applied against the pressure region 2, to hold the bandage firmly in place while the pull-tab region 5 of the protective film element 4 is pulled until all of the seal 6 is pulled apart, detaching the backing element 1 from the film element 4. Preferably, the protective film 4 is pulled by the pull-tab region 5 until the protective film 4 is removed.

The powdered hemostatic substance 3 is now in contact with the wound 7 and because of its powdered or finely divided state, the resulting large surface area of dry hemostatic hydrates and activates rapidly from blood at the wound site. Within several minutes a clot forms, sealing the wound 7.

The backing element 1 can be rectangular, oval, round, or any other shape and can be made from a conventional, non-bioabsorbable material such as an alginate (for example, calcium alginate), silicone, or plastic patch, or it can be an bioabsorbable material of protein, carbohydrate, or other material such as a glycolic acid polymer or a glycolic acid/lactic acid copolymer that can be broken down by the body into non-toxic components.

The protective film element 4 can be any material such as polyethylene or other plastic that can be sealed to the backing, and that provides a confining, protective environment for the powdered hemostatic substance 3.

The seal 6 can be any physiologically acceptable adhesive substance, or a seal created by heat or pressure applied to the film element 4 and/or backing element 1 that will retain the hemostatic powder 3, and bind the elements 1, 4 in an easily removable manner.

The hemostatic agent(s) can be proteins such as fibrinogen and thrombin, carbohydrates such as a suitable algal polymer (an algae-derived polysaccharide, for example) or chitosan (Pusateri et al 2003), minerals (Alam et al 2004), or other known hemostatic substances.

All components should be suitable for sterilization by any known means, such as by gamma radiation.

### EXAMPLE

A fibrin bandage was constructed as follows:

The backing was a 10 cm² Polysorb® (polyester) 2 mm thick felt (U.S. Surgical Corp.). Lyophilized salmon fibrinogen (19 mg/cm²) and thrombin (50 U/cm²) were mixed and applied as a finely ground powder to the Polysorb® backing. The powder was applied to all of the Polysorb® backing except for a 10×2 cm area on one end, the pressure region.

The fibrinogen and thrombin formed a loose covering of powder ranging from 0.5 to 0.8 cm thick.

One end of a 10×20 cm polyethylene sheet was used to cover the powder. The sheet was heat-sealed to the Polysorb® backing around the edge of the powder, surrounding and containing the powder. The remaining polyethylene sheet was then folded back over the enclosed powder, leaving a 2 cm portion of the sheet extending beyond the bandage as a “pull-tab”.

The bandage was placed, with the polyethylene side down, on a cellulose sponge that had been soaked in distilled water. Hand pressure was applied to the backing, and firm pressure was applied to the pressure region while at the same time, the pull-tab was pulled to remove the entire polyethylene sheet. The powder hydrated rapidly and within 1 to 3 minutes formed a firm clot or gel.

The composition of the hemosatic powder can be varied by one skilled in the art. For example, the powder may contain added calcium, stabilizers, detergents, or other additives, or may be only fibrinogen, if there is sufficient thrombin and calcium available from the patient's blood.

In a further modification, hemostatic substances such as thrombin and fibrinogen can be separated from each other by an adhesive, heat, or pressure seal in the area enclosed by the plastic film. These components would be in contact with each other only when the film is removed.

### REFERENCES

Alam H B, Chen Z, Jaskille J, Irenio R, Querol L C, Koustova E, Inocencio R, Conrad R, Seufert A, Ariaban N, Toruno K, Rhee P. 2004. Application of a zeolite hemostatic agent achieves 100% survival in a lethal model of complex groin injury in swine. J. Trauma: 974-82.
Bell et al. U.S. Pat. No. 5,800,372, Sep. 1, 1998.
Holcomb J B, Pusateri A E, Harris R A, Reid T J, Beall D, Hess J R, MacPhee M J. 1999 Dry fibrin sealant dressings reduce blood loss, resusitation volume, and improve survival in hypothermic, coagulopathic swine with Grade V liver injuries. J. Trauma (47):233-42.
Hursey F X, 2005. US Patent Application #20050074505. Apr. 7, 2005.
MacPhee et al. U.S. Pat. No. 6.054.122. Apr. 25, 2000
MacPhee et al. U.S. Pat. No. 6,197,325. Mar. 6, 2001
MacPhee et al. U.S. Pat. No. 6,762,336. Jul. 13, 2004
Michaud S E, Wang L Z, Korde N, Bucki R, Randhawa P K, Pastore J J, Falet H, Hoffmeister K, Kuuse R, Uibo R, Herod J, Sawyer E S. Janmey P A. 2002. Purification of salmon thrombin and its potential as an alternative to mammalian thrombins in fibrin sealants. Thrombosis Research 107:245-54.
Pusateri A E, McCarthy S J, Gregory K W, Harris R A, Cardenus L, MaManus A T, Goodwin C W. 2003. Effect of a chitosan-based hemostatic dressing on blood loss and survival in a model of severe venous hemorrhage and hepatic injury in swine. J. Trauma 57(1): 177-182.
Rothwell S W, Reid T J, Dorsey J, Flournoy W S, Bodo M, Sawyer E S, Janmey P A. 2005. A salmon thrombin-fibrin bandage controls arterial bleeding in a swine aortotomy model. J. Trauma 59(1):143-149.
Sawyer, E S et al. U.S. Pat. No. 6,007,811. Dec. 28, 1997.
Sawyer, P N. U.S. Pat. No. 4,390,519. Jun. 28, 1983.
Sondeen J L, Pusateri A E, Coppes V G, Gaddy C E, Holcomb J B. 2003. Comparison of 10 different hemostatic dressings in an aortic injury. J. Trauma 54:280-85.
Wang L Z, Gorlin J, Michaud S E, Janmey P A, Goddeau P R P, Kuuse R, Uibo R, Adams D, 2000. Purification of salmon clotting factors and their use as tissue sealants. Thrombosis Research 100:537-548.

1. A hemostatic compression bandage, comprising:
a flexible backing element;
a powdered hemostatic substance; and
a flexible film element;
wherein:
the backing element includes a first containment region;
the film element includes a second containment region corresponding to the first containment region;
the film element is removably attached to the backing element so as to contain the hemostatic substance between the backing element and the film element within a volume defined by the first containment region and the second containment region;
the film element includes a pull-tab region located outside of the second containment region and at a first side of the bandage; and
the pull-tab region has sufficient length to be folded back across the second containment region to extend beyond a second side of the bandage.

2. The hemostatic compression bandage of claim 1, wherein the backing element includes a pressure region located outside of the first containment region on at least one side of the bandage;

3. The hemostatic compression bandage of claim 2, wherein the pressure region is located on a side of the bandage that is opposite the second side of the bandage.

4. The hemostatic compression bandage of claim 1, wherein the backing element is fabricated from a non-bioabsorbable material.

5. The hemostatic compression bandage of claim 4, wherein the backing element includes at least one of an alginate, silicone, plastic, and cotton gauze.

6. The hemostatic compression bandage of claim 5, wherein the backing element includes calcium alginate.

7. The hemostatic compression bandage of claim 1, wherein the backing element is fabricated from a bioabsorbable material.

8. The hemostatic compression bandage of claim 4, wherein the backing element includes at least one of a glycolic acid polymer and a glycolic acid/lactic acid copolymer.

9. The hemostatic compression bandage of claim 1, wherein the backing element includes polyester felt.

10. The hemostatic compression bandage of claim 1, wherein the hemostatic substance includes a protein.

11. The hemostatic compression bandage of claim 10, wherein the hemostatic substance includes at least one of fibrinogen and thrombin.

12. The hemostatic compression bandage of claim 11, wherein the hemostatic substance includes at least one of lyophilized salmon fibrinogen and thrombin.

13. The hemostatic compression bandage of claim 1, wherein the hemostatic substance includes a carbohydrate.

14. The hemostatic compression bandage of claim 13, wherein the hemostatic substance includes at least one of an algal polymer and chitosan.

15. The hemostatic compression bandage of claim 14, wherein the hemostatic substance includes an algae-derived polysaccharide.

16. The hemostatic compression bandage of claim 1, wherein the hemostatic substance includes a mineral.

17. The hemostatic compression bandage of claim 1, further comprising powdered calcium, added to the hemostatic substance.

18. The hemostatic compression bandage of claim 1, wherein the film element includes plastic.

19. The hemostatic compression bandage of claim 18, wherein the film element includes polyethylene.

20. The hemostatic compression bandage of claim 1, wherein the film element is removably attached to the backing element by a seal that defines a perimeter of the first containment region and the second containment region.

21. The hemostatic compression bandage of claim 20, wherein the seal includes an adhesive substance.

22. The hemostatic compression bandage of claim 20, wherein the seal is a bond formed by at least one of heat and pressure.

23. The hemostatic compression bandage of claim 1, wherein the backing element, the powdered hemostatic substance, and the film element are fabricated from materials that are suitable for sterilization.

24. The hemostatic compression bandage of claim 23, wherein the backing element, the powdered hemostatic substance, and the film element are fabricated from materials that are suitable for sterilization by gamma radiation.

25. The hemostatic compression bandage of claim 1, wherein the backing element includes an adhesive substance around a periphery, facing the film element and outside of an outer periphery of the film element.

26. A method of treating an open wound, comprising:
placing the hemostatic compression bandage of claim 25 over the wound, with the film element pressed against the wound;
applying pressure against the backing element over the wound;
grasping the pull-tab region of the film element;
pulling the film element out from under the powdered hemostatic substance and away from the backing element, allowing the powdered hemostatic substance to come into contact with the wound; and
pressing the adhesive substance against skin around the wound, thereby adhering the backing element to the skin.

27. The method of claim 26;
wherein the backing element includes a pressure region located outside of the first containment region on at least one side of the bandage, and
further comprising applying sufficient pressure against the pressure region to hold the bandage in place as the film element is pulled.

28. A method of treating an open wound, comprising:
placing the hemostatic compression bandage of claim 1 over the wound, with the film element pressed against the wound;
applying pressure against the backing element over the wound;
grasping the pull-tab region of the film element; and
pulling the film element out from under the powdered hemostatic substance and away from the backing element, allowing the powdered hemostatic substance to come into contact with the wound.

29. The method of claim 28, further comprising applying pressure against the backing element until the powdered hemostatic substance causes blood at the surface of the wound to clot.

30. The method of claim 28,
wherein the backing element includes a pressure region located outside of the first containment region on at least one side of the bandage, and
further comprising applying sufficient pressure against the pressure region to hold the bandage in place as the film element is pulled.
